(19) United States
(12) Patent Application Publication
(10) Pub. No.: US 2017/0086785 A1
(43) Pub. Date: Mar. 30, 2017

(54) SYSTEM AND METHOD FOR PROVIDING TACTILE FEEDBACK VIA A PROBE OF A MEDICAL IMAGING SYSTEM

(71) Applicant: General Electric Company, Schenectady, NY (US)

(72) Inventor: Steinar Bjaerum, Horten (NO)

(21) Appl. No.: 14/871,801

(22) Filed: Sep. 30, 2015

(51) Int. Cl. A61B 8/00; A61B 8/13; A61B 8/08

(52) U.S. Cl. CPC A61B 8/4254; A61B 8/4444; A61B 8/5207; A61B 8/461; A61B 8/467; A61B 8/13; A61B 8/5215

(57) ABSTRACT

Methods and systems are provided for providing tactile feedback via a probe of an imaging system. In one embodiment, a method comprises acquiring ultrasound data with a probe, generating an image based on the ultrasound data, determining whether the probe is in a correct position to acquire a desired region of interest, and providing a first tactile feedback through the probe in response to a determination that the probe is in the correct position. In this way, a user may increase the accuracy of probe positioning, thereby producing more accurate medical images for diagnostic purposes.

### FIELD

Embodiments of the subject matter disclosed herein relate to medical imaging and more particularly, to methods and systems for acquiring ultrasound images.

### BACKGROUND

An ultrasound imaging system typically includes an ultrasound probe that is applied to a patient's body and a workstation or device that is operably coupled to the probe. The probe may be controlled by an operator of the system and is configured to transmit and receive ultrasound signals that are processed into an ultrasound image by the workstation or device. A user positions the probe to acquire a desired region of interest (ROI) (e.g., a desired tissue or body region to be imaged) in a desired scan plane. For example, by viewing real-time images of the acquired ultrasound data on a display of the ultrasound imaging system, a user may translate, rotate, and/or pivot the probe to adjust the probe into a correct position for imaging the desired scan plane of the desired region of interest.

However, the inventors herein have recognized challenges with such positioning methods. For example, manually finding the correct position on a patient via viewing the displayed images alone may be difficult, time-consuming, and result in less accurate positioning, especially for unskilled users.

### BRIEF DESCRIPTION

In one embodiment, a method comprises acquiring ultrasound data with a probe, generating an image based on the ultrasound data, determining whether the probe is in a desired position to acquire a desired region of interest, and providing a first tactile feedback through the probe in response to a determination that the probe is in the desired position. In this way, a user may position the probe more accurately and easily.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an example ultrasonic imaging system according to an embodiment of the invention.

FIG. 2 shows a flow chart illustrating a method for outputting tactile feedback via a probe in response to undesired and desired probe placement.

FIG. 3 shows a flow chart illustrating a method for a user wielding a tactile feedback emitting probe.

### DETAILED DESCRIPTION

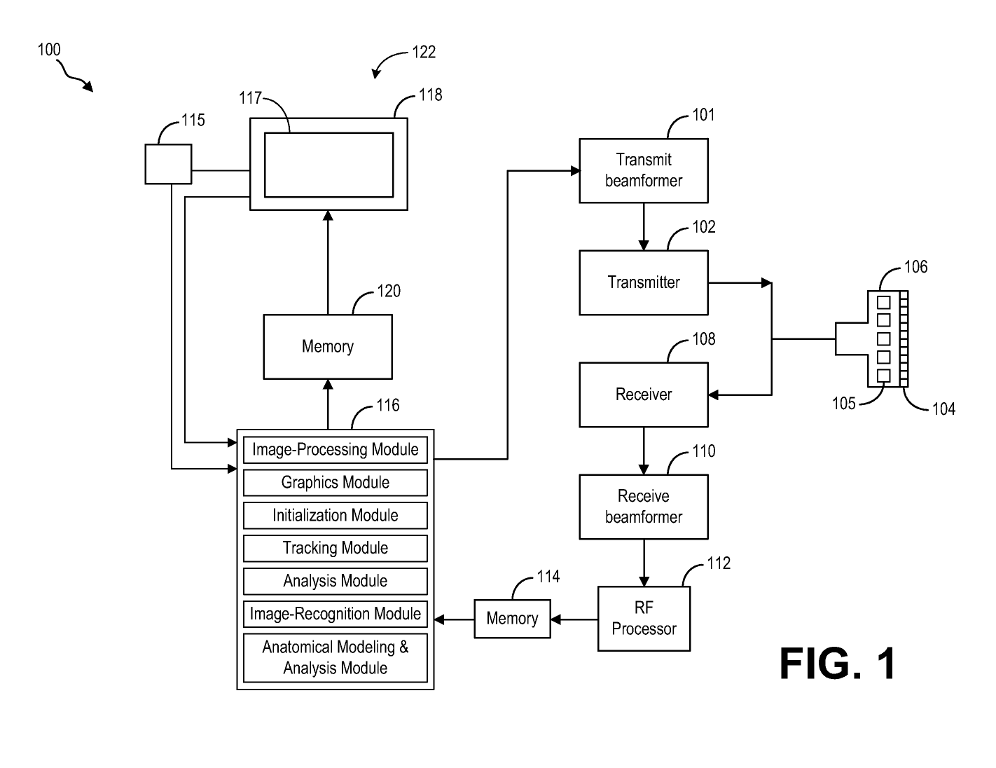

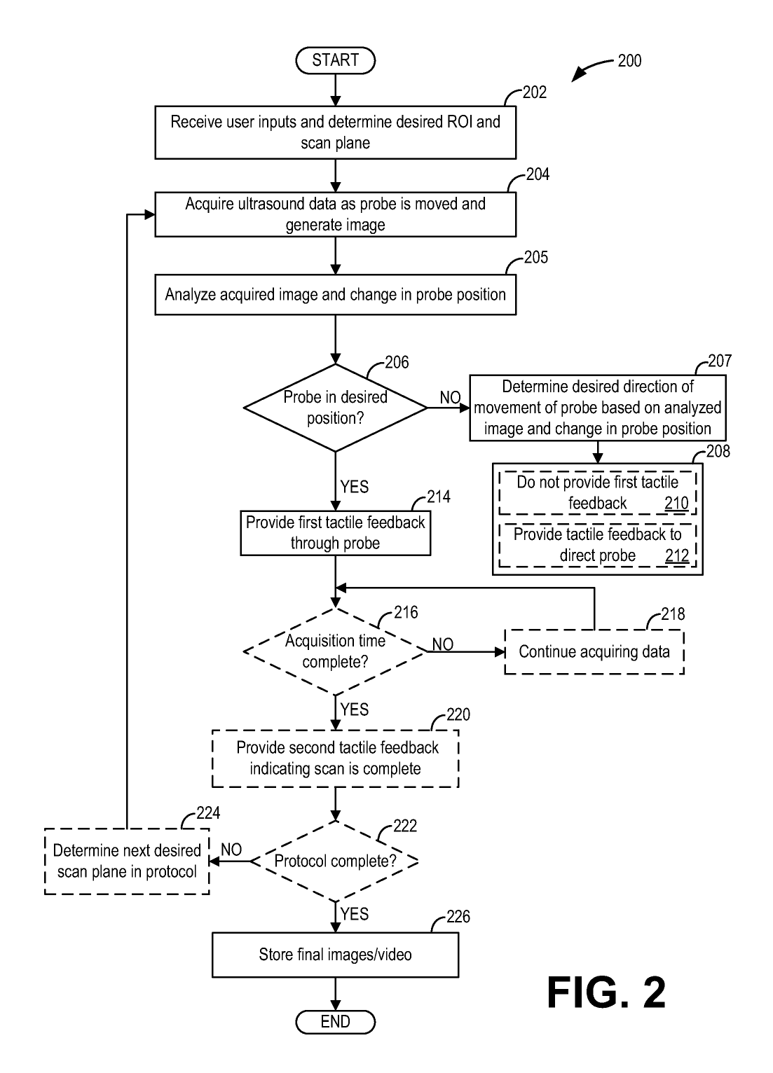

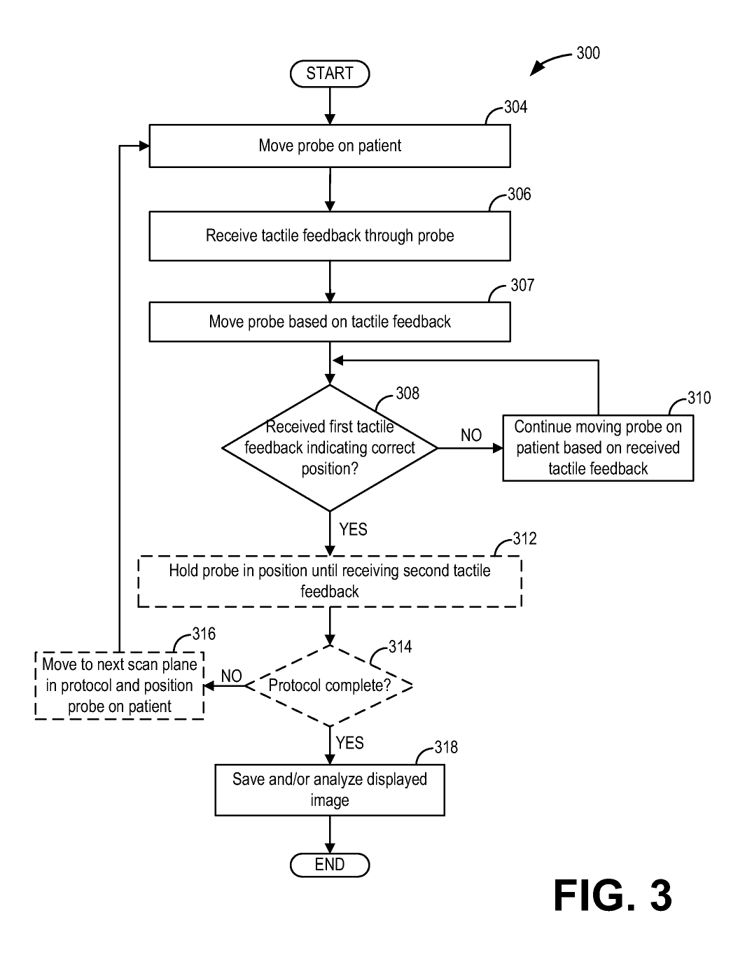

The following description relates to various embodiments of an imaging system, such as the ultrasound imaging system shown in FIG. 1. In particular, systems and methods are provided for providing tactile feedback via a probe of the imaging system, such as the probe shown in FIG. 1. FIG. 2 presents a method for outputting tactile feedback via the probe in response to probe position. As used herein, position may include one or each of probe location and orientation, where location is a specific coordinate, or place, on/in a patient, and orientation is the angle at which the probe is placed. The tactile feedback emitted (e.g., the intensity, pattern, duration or mode of tactile feedback) may be based on the data used to generate the image, as well as outputs from probe motion sensors responsible for tracking a change in probe position. A user may receive different types of tactile feedback depending on whether the probe is in a desired position or not. FIG. 3 shows a methodology from the user perspective for handling and responding to a tactile feedback emitting probe in order to achieve desired probe placement for accurate imaging. FIG. 3 further entails a method for capturing multiple scan planes when the protocol requires multiple planes of view.

FIG. 1 illustrates a block diagram of an ultrasound imaging system 100 according to one embodiment. As shown, the system 100 includes multiple components. The components may be coupled to one another to form a single structure, may be separate but located within a common room, or may be remotely located with respect to one another. For example, one or more of the modules described herein may operate in a data server that has a distinct and remote location with respect to other components of the system 100, such as a probe and user interface. Optionally, in the case of ultrasound systems, the system 100 may be a unitary system that is capable of being moved (e.g., portably) from room to room. For example, the system 100 may include wheels or be transported on a cart.

In the illustrated embodiment, the system 100 includes a transmit beamformer 101 and transmitter 102 that drives an array of elements 104, for example, piezoelectric crystals, within a diagnostic ultrasound probe 106 (or transducer) to emit pulsed ultrasonic signals into a body or volume (not shown) of a subject. Furthermore, the probe is outfitted with one or more actuators 105 capable of receiving signals from a system controller 116, as described further below, in order to output tactile feedback to the user. The elements 104, the one or more actuators 105, and the probe 106 may have a variety of geometries. The ultrasonic signals emitted by the elements 104 are back-scattered from structures in the body, for example, blood vessels and surrounding tissue, to produce echoes that return to said elements 104. The echoes are received by a receiver 108. The received echoes are provided to a beamformer 110 that performs beamforming and outputs an RF signal. The RF signal is then provided to an RF processor 112 that processes the RF signal. Alternatively, the RF processor 112 may include a complex demodulator (not shown) that demodulates the RF signal to form IQ data pairs representative of the echo signals. The RF or IQ signal data may then be provided directly to a memory 114 for storage (for example, temporary storage).

The system controller (e.g., electronic controller) 116 of the system 100 includes a plurality of modules, which may be part of a single processing unit (e.g., processor) or distributed across multiple processing units. The system controller 116 is configured to control operation of the system 100. For example, the system controller 116 may include an image-processing module that receives image data (e.g., ultrasound signals in the form of RF signal data or IQ data pairs) and processes image data. For example, the image-processing module may process the ultrasound signals to generate slices or frames of ultrasound information (e.g., ultrasound images) for displaying to the operator. In system 100, the image-processing module may be configured to perform one or more processing operations according to a plurality of selectable ultrasound modalities on the acquired ultrasound information. By way of example only, the ultrasound modalities may include color-flow, acoustic radiation force imaging (ARFI), B-mode, A-mode, M-mode, spectral Doppler, acoustic streaming, tissue Doppler module, C-scan, and elastography. The generated ultrasound images may be two-dimensional (2D) or three-dimensional (3D). When multiple two-dimensional (2D) images are obtained, the image-processing module may also be configured to stabilize or register the images.

Acquired ultrasound information may be processed in real-time during an imaging session (or scanning session) as the echo signals are received. Additionally or alternatively, the ultrasound information may be stored temporarily in the memory 114 during an imaging session and processed in less than real-time in a live or off-line operation. An image memory 120 is included for storing processed slices of acquired ultrasound information that are not scheduled to be displayed immediately. The image memory 120 may comprise any known data storage medium, for example, a permanent storage medium, removable storage medium, and the like. Additionally, the image memory 120 may be a non-transitory storage medium.

In operation, an ultrasound system may acquire data, for example, volumetric data sets by various techniques (for example, 3D scanning, real-time 3D imaging, volume scanning, 2D scanning with probes having positioning sensors, freehand scanning using a voxel correlation technique, scanning using 2D or matrix array probes, and the like). Ultrasound images of the system 100 may be generated from the acquired data (at the system controller 116) and displayed to the operator or user on the display device 118.

The system controller 116 is operably connected to a user interface 122 that enables an operator to control at least some of the operations of the system 100. The user interface 122 may include hardware, firmware, software, or a combination thereof that enables a user (e.g., an operator) to directly or indirectly control operation of the system 100 and the various components thereof. As shown, the user interface 122 includes a display device 118 having a display area 117. In some embodiments, the user interface 122 may also include one or more input devices 115, such as a physical keyboard, mouse, and/or touchpad. In an exemplary embodiment, the display device 118 is a touch-sensitive display (e.g., touchscreen) that can detect a presence of a touch from the operator on the display area 117 and can also identify a location of the touch in the display area 117. The touch may be applied by, for example, at least one of an individual's hand, glove, stylus, or the like. As such, the touch-sensitive display may also be characterized as an input device that is configured to receive inputs from the operator. The display device 118 also communicates information from the system controller 116 to the operator by displaying the information to the operator. The display device 118 and/or the user interface 122 may also communicate audibly. The display device 118 is configured to present information to the operator during the imaging session. The information presented may include ultrasound images, graphical elements, user-selectable elements, and other information (e.g., administrative information, personal information of the patient, and the like). Additionally, the information presented may include confirmation of correct probe positioning. In one embodiment, the display area 117 may be outfitted with a visual probe position accuracy gauge that transitions from red, through yellow, to green as the probe moves from an incorrect scan plane to a correct scan plane. In other embodiments the system controller 116 may send probe positioning information simultaneously to the display area 117 and actuators 105 of the probe 106, so that the user receives a combination of visual feedback/instructions on the display area 117 while also receiving tactile feedback through the actuators 105 of the probe 106. Lastly, the display area 117 may be used to display a reference point at one edge of the displayed ultrasound image, as described further below.

In addition to the image-processing module, the system controller 116 may also include a graphics module, an initialization module, a tracking module, an analysis module, an image-recognition module, and/or an anatomical modeling & analysis module. The image-processing module, the graphics module, the initialization module, the tracking module, the analysis module, the image-recognition module, and the anatomical modeling & analysis module may coordinate with one another to present information to the operator during and/or after the imaging session. For example, the image-processing module may be configured to display an acquired image on the display device 118, and the graphics module may be configured to display designated graphics along with the ultrasound image, such as graphical outlines, which represent lumens or vessel walls in the acquired image. The image-processing and/or graphics modules within the system controller 116, may also be configured to generate a 3D rendering or image (not shown) of the entire vascular structure. At the same time, the tracking module may be coordinating with the image-recognition module and anatomical modeling & analysis module to determine how a change in probe position (e.g., a change in the location on the tissue and/or an orientation (e.g., angle) with respect to the tissue) affects the ultrasound image. The tracking module (and possibly other modules) may further be in communication with one or more motion sensors located within the probe (e.g., within a body of the probe), such as an accelerometer, gyroscope, magnetometer, or any combination therein, to improve positioning accuracy (increased accuracy is achieved when knowing how much probe movement results in a given change in the image). In one embodiment, image analysis may be executed in part by automatically detecting anatomical landmarks, and may use a computerized anatomical model (3D) (e.g., geometrical model) to calculate how to move the probe in order to obtain the desired probe position. Furthermore, stored images (such as, stored images in the memory 120, the memory 114, or a remote data server), and image-recognition may be utilized in an over-arching image analysis algorithm that makes determining probe position, as well as determining which direction the probe ought to move to be in the desired position, possible. It will be appreciated that image analysis of the generated and displayed images combined with known incremental changes to probe position enable the system to determine the position of the probe and further guide positioning of the probe toward a desired region of interest. The modules of the system controller 116 may be used to guide positioning of the imaging probe by analyzing how the generated and displayed image changes in response to a change in probe position. For example, based on a currently generated ultrasound image (based on data acquired via the probe), the system controller 116 instructs, by tactile (and possibly visual feedback via the display and/or audio feedback) feedback, the user to execute incremental changes in probe position in a specified direction.

Returning to the aforementioned tactile feedback emitting probe 106, the probe 106 is equipped with one or more actuators 105 to output tactile feedback to the user. As one example, the probe 106 may include multiple actuators 105 located peripherally around the handheld portion of the probe 106 that are configured to output tactile feedback to the user. Based on positioning of the probe 106, a plurality of tactile feedback outputs indicating correct/incorrect positioning will be administered to the user via the actuators 105 to aid in correct probe 106 positioning for the desired ultrasound protocol. In one embodiment, the tactile feedback emitted via the probe may mirror the aforementioned visual probe position accuracy gauge in that the type of feedback output may change as a user shifts from an incorrect to correct probe position. Accordingly, the disclosed system 100 may decrease human error that arises from misalignment of the probe's elements 104 through misalignment detection (i.e., detection of probe not in desired position) and tactile feedback correction. The disclosed system 100 may provide tactile feedback to indicate correct positioning of the probe on a tissue or patient. Outputting tactile feedback from the actuators 105 of the probe 106 to indicate correct positioning may be a more effective way of conveying correct positioning to the user compared to visual feedback via the display since it may be more immediate and may not require the user to direct their attention to the display area 117 for visual conformation of positioning. Instead, a user may focus on the probe and patient without having to rely on the image displayed on the display screen. Another advantage to tactile feedback output from a probe is the possibility of providing the user, especially a less experienced user, with physical feedback instructing how to move the probe and manipulate scan plane positioning (via changing the probe orientation on a surface of the tissue being imaged).

The ultrasound system knows, from the internal wiring and physical assembly of the probe, how the acquired image plane is rotated relative to the probe. The probe 106 may have a physical mark such as a knob to indicate a reference point. In one example, the physical reference point mark may include a light emitting diode (LED). The reference point may be displayed on the display area 117 at one edge of the displayed ultrasound image (not shown). For example, if the reference point of the probe 106 is directed towards a patient's head, then the reference mark on the display area 117 indicates which edge of the image is directed toward the patient's head. In this way, the physical reference point with accompanying display of the generated image, aids the operator when making a mental image of how to manipulate the probe in order to obtain the desired scan plane for the desired region of interest. The tactile feedback system can similarly utilize this information. In order for the system 100 to give correct directional feedback (for positioning the probe in a desired position) to the user via outputting tactile feedback via the probe, it must monitor the position of the probe's reference point relative to the anatomy of the patient as the probe moves around on the patient's tissue. For example, if the system determines via image analysis that the probe should be moved in a direction opposite to the reference point, then the actuators located at the opposite side relative to the physical mark may be triggered, indicating that the probe should be moved in this direction. Similarly, appropriately located actuators may be triggered to indicate, for instance, clockwise rotation. In another embodiment, there may be triggering of appropriately located actuators to indicate upwards tilting of the probe. In this way, the reference point may be used to trigger subsets of actuators in order to impart which way the probe needs to move in a three dimensional space (encompassing linear, rotational, pivotal and tilting movements), to approach/arrive at a desired location of the ROI.

The system controller 116 may continuously analyze the ultrasound image to determine how the ultrasound probe 106 is positioned/oriented relative to anatomical structures. The system controller 116, any possible internal motion sensors of the probe 106, and the actuators 105, are all interfacing while the ultrasound image is visible on the display device 118. In this way, the user has visual feedback via the display area 117 and haptic feedback via the actuators 105 within the probe 106. Additionally, this method ensures that the tactile feedback received by the user is “live”, so that there is no break between what the user is viewing on the display area 117 and the tactile feedback received from the actuators 105 of the probe 106. As used herein, internal motion sensors, refers to one or more of an accelerometer, gyroscope, and magnetometer, which may work independently or together to gather information about movement of the probe (such as linear or rotational movement, which when combined together provide three dimensional positioning information of the probe). It will be appreciated that positioning information provided by any internal motion sensors of the probe may increase the efficacy of the tactile feedback system by allowing the controller to determine how much probe movement corresponded to a given change in the ultrasound image. It will also be appreciated that while tactile feedback is often discussed with 2D examples, that a tactile feedback system may also be applicable to 3D imaging.

In one embodiment, the system controller 116 has an image recognition module comprised of stored image data, and an anatomical modeling & analysis module. In this embodiment, signals from the probe 106 are sent to the system controller 116 where they are then compared to stored images in order to determine if the probe 106 is placed correctly. Additionally, the modules within the controller analyze how the ultrasound image has changed with respect to probe position change in order to determine if the probe is positioned correctly, and if not, which way the probe must shift position to be in the desired position. A signal is then sent back to the actuators 105 of the probe 106 to output tactile feedback to the user, indicating correct/incorrect probe position and/or which direction to move the probe toward the correct position for acquiring the desired image (e.g., region of interest and scan plane). For example, incorrect positioning signals may be localized to a subset of actuators in order to indicate to the user which direction to move the probe to approach or arrive at the desired position. As with the previous embodiment, the ultrasound data is simultaneously processed to provide an image on the display area 117 and tactile feedback through the probe 106, so that the tactile feedback corresponds to what the user is viewing. It will be appreciated that in protocol based imaging, the system includes instructions stored within a memory of the controller for instructing the user to position the probe for acquiring a specific scan plane. In this way, the system controller 116 can compare the incoming probe 106 signals to a specific set of images that correspond with the protocol being executed. For example, if the user is performing an ultrasound of the renal arteries, they may communicate this to the system controller 116 via an input device 115 and/or user interface 122. The system 100 then knows from its stored instructions that the image-recognition module should be comparing incoming ultrasound data from the probe 106 to stored images that meet all relevant criteria, such as: ultrasound mode being used (Doppler or B-mode), desired region of interest (and nearby anatomical features for when the probe is not in the desired position), desired scan plane (and possible incorrect scan planes for a particular ROI and neighboring anatomy when in a specific ultrasound mode). The system controller 116 may then limit the images it searches within the image recognition module to the appropriate anatomical features, scan planes, and ultrasound mode. In this way, the system controller 116 may be able to send tactile feedback signals to the probe 106 in a faster and more efficient manner.

In yet another embodiment, the system 100 may be outfitted with each of internal motion sensors (i.e., one or more of an accelerometer, gyroscope, and or magnetometer), and a number of image analysis modules, including but not limited to an image recognition module, a tracking module, and an anatomical modeling & analysis module (as described above). For example, if the user is performing an ultrasound of the renal arteries, they may communicate to the system controller 116 via an input device 115 and/or user interface 122. The user may then indicate they are taking an oblique approach, and further specify that the system 100 is in Doppler mode. An oblique approach requires the probe 106 be held at a 45 degree angle to achieve the desired scan plane. In this particular example the system controller 116 may compare the incoming probe data to images of the renal artery from an oblique approach using Doppler mode and then output tactile feedback signals via actuators 105 to the user in order to get the user to place the probe 106 in the correct position (both gross location and orientation). While the user is moving the probe from one position to the next, the system may be constantly analyzing and displaying new (e.g., updated) ultrasound images based on incoming data from the probe 106 to the display area 117. With the help of internal motion sensors, a tracking module within the system controller 116 may more accurately track precisely how much probe movement resulted in a given change in the ultrasound image. It will be appreciated that the motion sensor data is still being analyzed alongside imaging data, and that the internal motion sensors merely supplement imaging analysis data to increase the accuracy of positioning. In this way, internal motion sensors may increase the accuracy of tactile feedback provided to the user via the probe. For example, the strength of the output tactile feedback signal may be one adjustable parameter of the tactile feedback. In one embodiment, the strength of the signal output may indicate how far from the desired position a user is, so that a weak signal means the user is close to the desired position and a strong signal indicates the probe is a greater distance away from the desired position. In this way, the strength (e.g., magnitude) of the tactile feedback signal output via the probe may decrease as a user moves the probe closer to the desired positioning (e.g., location and orientation) for acquiring the desired region of interest and scan plane. In another embodiment, the strength of the tactile feedback signal may increase as the probe gets closer to the desired positioning. By incorporating internal motion sensors to the probe 106, the system 100 may be able to output more accurate tactile feedback instructions to the user, thereby increasing the efficacy of the entire system. A final affirmative signal may be sent to the user via tactile feedback through the probe indicating that the positioning is correct once the controller determines the probe is in the desired position. It will be appreciated that the modules listed, and the internal motion sensors may operate concertedly, independently, at different points in the procedure, throughout the entire procedure, etc. As with the previous two embodiments, the user will be receiving tactile feedback that corresponds with the image on the display device 118.

In one embodiment, the actuators 105 responsible for outputting tactile feedback may be peripherally spaced in a continuous ring around the portion of the probe 106 that the user grips. There may be multiple types of actuators to output various types of tactile feedback. Tactile feedback may present as any combination of vibrations (variable pulses differing in pattern, duration, intensity, etc.), temperature (heating and cooling), or force feedback (robotic manipulators applying forces/pulses against the probe 106 in an external direction from the central longitudinal axis of the probe toward the external user grip). The types of tactile feedback may be variable depending on user preference, the type of ultrasound being performed (mode, 2D, 3D, etc.), and whether the probe is in the desired (e.g., correct) or undesired (e.g., incorrect) position.

Turning to FIG. 2, a flow chart of a method 200 for outputting tactile feedback via a probe outfitted with actuators used in an imaging system is shown. The method 200 and other methods disclosed herein (e.g., method 300 shown in FIG. 3) may be performed with an imaging system, such as the ultrasound imaging system 100 shown in FIG. 1. Thus, FIGS. 2-3 will be described further below according to an exemplary embodiment where the methods are performed with the ultrasound imaging system 100 shown in FIG. 1. More specifically, method 200 and the other methods disclosed herein may be executed by a controller of the ultrasound imaging system (such as controller 116 shown in FIG. 1) according to instructions stored on a non-transitory memory of the system (e.g., such as memory 120 shown in FIG. 1) in combination with the various signals received at the controller from the system components and actuator signals sent from the system controller to the probe. However, according to other embodiments, the methods 200 and 300 may also be performed with other ultrasound imaging systems or with different medical imaging devices.

Method 200 begins at 202, where the ultrasound system receives user inputs and determines a desired region of interest (ROI) (i.e., anatomical structure to be imaged) and scan plane. The system controller may receive this information from a user interface such as a keyboard, mouse, tablet, etc. For example the system controller may receive information from the user via mouse clicks in a drop down menu within the display area of the system that a liver is being imaged from a parasagittal scan plane in B-mode. In an alternative embodiment, the ultrasound system may not be dependent on receiving user inputs and may instead be running an imaging protocol. In protocol based imaging, the system is capable of setting itself up and instructing the operator to position the probe for acquisition of a specific scan plane (as opposed to the operator telling the system which scan plane is going to be acquired). As such, in this alternative embodiment, the method at 202 may include receiving the scan plane for the ROI from the system controller (e.g., via the display or another user interface).

At 204, the method includes acquiring ultrasound data and generating an image. For example, once the probe is positioned on an object surface, the controller signals the probe to emit pulsed ultrasonic signals into a body or volume of a subject, as described above with reference to FIG. 1. The ultrasonic signals are back-scattered from structures in the body, producing echoes that return to the elements of the probe. The echoes are received by a receiver (such as receiver 108 show in FIG. 1), then a beamformer (such as beamformer 110 shown in FIG. 1), which outputs a RF signal. The RF signal may then be transmitted to a RF processor (such as RF processor 112 shown in FIG. 1) which outputs RF signal data, or if the RF processor contains a complex demodulator, IQ signal data is output. Once ultrasound data is output, it can be processed by the system controller (such as system controller 116 shown in FIG. 1) to generate an image on the display device (such as display device 118 shown in FIG. 1) for user viewing. For example, the signal data acquired up until this point is then processed and analyzed by the system controller (e.g., such as controller 116 shown in FIG. 1) in order to produce an ultrasound image. The system controller may include an image-processing module that receives the signal data (e.g., image data) acquired thus far and processes the received image data. For example, the image-processing module may process the ultrasound signals to generate slices or frames of ultrasound information (e.g., ultrasound images) for displaying to the operator. In one example, generating the image may include determining an intensity value for each pixel of a display screen (e.g., display area 117 shown in FIG. 1) based on the received image data (e.g., 2D or 3D ultrasound data). As such, the ultrasound images may be two-dimensional (2D) or three-dimensional (3D) depending on the mode of ultrasound being used (e.g., color-flow, acoustic radiation force imaging (ARFI), B-mode, A-mode, M-mode, spectral Doppler, acoustic streaming, tissue Doppler module, C-scan, and elastography).

At 205, the method continues by analyzing the acquired image and change in probe position. As previously described with reference to FIG. 1, the system controller may be outfitted with a number of image analysis related modules, as well as a tracking module. The system controller will already be aware of the desired anatomical structure to be imaged (e.g., ROI), and the desired scan plane. As the probe is moved along a patient's body the incoming ultrasound data will be analyzed and the change in passing anatomical features will be processed. With this information regarding changing anatomical landscape and the change in direction/orientation of the probe's reference point (relative to the patient's anatomy), the system controller will be able to determine which direction the probe is moving (in addition to the identity of the underlying anatomical features passing by). For example, the user may attempt to image the spleen. The user may initially start acquiring ultrasound data along the central line of the patient atop the pancreas. As the user translates the probe laterally in the direction of the spleen, passing anatomical features may include the left kidney, stomach, and large intestine. The system controller may contain instructions for utilizing image-analysis modules such as an anatomical modeling & analysis module. The system controller may then piece together a picture of the changing anatomical landscape in order to infer that the probe is approaching the desired region of interest (i.e., spleen). Secondarily to this primary tracking feature, an embodiment may include a probe that includes internal motion sensors (one or more of a gyroscope, accelerometer, and/or magnetometer) which may determine how much probe movement resulted in a given change of the generated and displayed image. Direct quantification of three dimensional probe movement (provided by an accelerometer, gyroscope and possibly magnetometer in conjunction with one another) compared to changes in the generated ultrasound image may increase the accuracy of positioning the probe by allowing the system to anticipate approaching anatomical features, and by extension, timing of output signals, based on acceleration and linear movement. Furthermore, this analysis in conjunction with knowing which direction the probe's reference point is facing may allow the system controller to know which way and where the probe is oriented with respect to the patient. In response to the system controller knowing where the probe is in a given anatomical landscape, the system controller may then be able to output tactile feedback to specific subsets of actuators to direct a user how to move the probe to acquire the desired ROI and scan plane (as described in greater detail below with reference to 207).

At 206, the method includes determining if the probe is in the desired position to acquire the desired ROI and scan plane. Determining whether the probe is in the desired position to acquire the desired region of interest may include performing image analysis of the generated image to determine whether the generated image substantially matches an expected image for the desired region of interest. As used herein, “substantially matches” means that the generated image from the ultrasound data matches the desired image (which may be a 2D model or 3D geometrical model) by a predetermined threshold percentage (e.g., generated image matches model by 90%). Additionally, as used herein, “expected image” may be based on one or more of stored image data and/or a geometrical model, wherein the geometrical model is a computerized anatomical 3D model of the image at the desired position. For example, image analysis may consist of detecting anatomical landmarks in the acquired ultrasound image, and comparing or matching the landmarks to a geometrical 3D model (and/or stored image data) of the anatomical structure being imaged. If the analyzed data (i.e., generated image) differs from the expected image, either by the probe being in the wrong location, the probe being held at the wrong orientation, or both, then the system determines the probe position to be incorrect and the method moves to 207. It will be appreciated that the probe may constantly be cycling through the methods at 204-207, that is, analyzing incoming data and probe position may be an on-going, real-time process.

At 207, the method continues by determining the desired direction of movement of the probe, based on the analyzed image and the change in probe position. Having acquired ultrasound data as the probe is moved at 204, identified key anatomical features at 205, and determined that the probe is not in the desired position at 206, the system controller now analyzes the most recent changes in probe position in order to orient the probe with respect to the anatomy of the patient. Image analysis may include analyzing the current image, a previously acquired image (e.g., the image generated at the previous probe position before it was moved to the current position), and comparing the two generated images. Having oriented the probe to the anatomy of the patient, and knowing where the desired region of interest is with respect to the current undesired position, the system controller may send a signal to a specific subset of actuators in order to produce tactile feedback through the subset of actuators. Activation of a localized number of actuators to output tactile feedback confers to the user which direction to move the probe to approach/reach the desired position. Determining the direction of movement needed to arrive at the desired position, and determining which activators to activate, may be at least partially based on monitoring the position of the probe's reference point relative to the anatomy of the patient as the probe moves along the patient. For example, the user may wish to image a spleen (desired scan plane arbitrary for this example), and may begin acquiring data from the central line of the patient, above the liver. The user translates the probe across the patient's abdomen in the lateral direction toward the spleen, and for this example, is presently positioning the probe above the stomach. In one embodiment, the system controller analyses the resulting change in the generated image (due to the change in the probe position), to determine that the probe is being moved in a lateral direction toward the spleen, but is presently above the stomach (at the same time the system controller is tracking where the reference point is relative to the anatomy of the patient). Knowing from previous user inputs or an imaging protocol that the objective is to image the spleen, and knowing that the probe is currently only above the stomach, but moving in the correct direction to eventually reach the spleen (from previously described image analysis), the system controller is then able to determine that the desired direction of movement is a lateral one, toward the spleen. The system controller may then determine where the reference point is relative to this desired direction of lateral movement, and use this positioning information to determine which activators to signal (relative to reference point). The system controller may then determine if and how to provide tactile feedback to guide the user toward the desired location. For example, the reference point may already be oriented toward the spleen, and so the system controller determines the actuators located in the same region of the probe as the reference point ought to be activated. It will be appreciated that alternative embodiments may include a probe that is additionally outfitted with internal motion sensors, in order to more accurately determine just how much probe movement resulted in a change in the generated and displayed ultrasound image.

The method continues to 208 which includes two possible methods, 210 and 212. At 208 either method 210 or 212, or both 210 and 212, may be carried out. At 210, the probe does not provide tactile feedback in response to the controller determining the probe is not in the desired position. For example, the system may be configured to only provide tactile feedback via the probe in the event of correct placement. As used herein, “correct” placement may be used to refer to the probe being in the desired position, as determined by the controller based on received user inputs and/or a known ROI and scan plane for the object being imaged. As another example, the system may be configured to only provide tactile feedback via the probe if the user manually requests tactile feedback. In one embodiment the user may manually request tactile feedback by interacting with the display area of the imaging device. The display area of the imaging device may have a host of user selectable elements which control probe, imaging, and/or user settings. Upon user input (via finger, stylus, keyboard, mouse, tablet, etc.), one of these user selectable elements may signal the system controller to assess the probe's position (location and/or orientation) and emit a tactile feedback signal. The described embodiment allows the user to receive tactile feedback only upon request. Alternatively, the system may be configured to only provide feedback if the probe has been stationary for a predetermined amount of time (e.g., 3 seconds). For example, the user may be in the correct gross location, but not angled correctly (i.e., incorrect orientation). The user may then be using the visual feedback of the display area (such as the display area 117 in FIG. 1) to attempt to angle the probe correctly. Simultaneously the system controller is processing incoming probe data, and is continuously determining the probe is not yet in the correct position. In this example, while the user is adjusting the angle of the probe, the probe will not emit tactile feedback because the user has not held said probe steady for a predetermined amount of time. As one example, the duration for which the probe must be stationary before it emits tactile feedback may be a system setting which the user may input to the controller (e.g., via a user interface of the system).

At 212, the method includes providing tactile feedback to direct the probe based on the determined desired direction of movement of the probe (as determined at 207). Having determined that the probe is not in the correct position (determination methods described above) and identified the desired direction of movement (e.g., translational, rotational, pivotal, etc.) of the probe to move toward the correct position, the system controller signals one or more actuators of the probe to output tactile feedback. For example, the system controller may have determined at 207 that the probe is oriented to the “left” of the desired ROI. The system controller may then signal a subset of actuators on the “right” side of the probe to output tactile feedback, in order to impart to the user that the probe must be moved to the “right” to approach/arrive at the desired ROI (note that “left” and “right” are arbitrary coordinate directions for the example provided). The tactile feedback may be in the form of vibration, temperature change, force feedback, etc. The type of tactile feedback may also be specific to incorrect positioning, that is, the user will receive a different type of tactile feedback if the probe is in an incorrect position than the type received if the probe is in the correct position. The different type of feedback may present in any number of ways. Incorrect position may be indicated by a different mode of tactile feedback. For example, tactile feedback indicating correct positioning may present as heat, whereas incorrect positioning may present as a vibration. Furthermore, the tactile feedback may be localized in such a way to indicate to the user which way the probe should move to be in the correct position. For example, if vibration is the mode of tactile feedback, and if the user has placed the probe to the right of the desired location, the probe may then vibrate on the left to indicate to the user to move the probe to the left. It will be appreciated that while the tactile feedback provided may impart to the user which direction to move the probe, that in an alternative embodiment, the tactile feedback may merely impart that the probe is in the incorrect position (providing no directional signal indicating which way the probe should be moved to be in the desired position). Different types of tactile feedback indicating correct/incorrect positioning may also present as variations of one mode of feedback. For example, if the mode is vibration, then the system controller will output different signals to the actuators of the probe, leading to different types of vibration, so that the user may know if they are in the correct/incorrect position. As with the previous example, tactile feedback may be localized to a portion of the probe to guide the user to the correct position. Furthermore, the strength/duration/pattern of these incorrect placement signals may vary depending on the placement circumstance. In one embodiment, the strength of the vibration may indicate how far off the user is, that is, a small vibration may indicate the probe is very close to the correct position and a large vibration may indicate the user is a considerable distance away from the correct position. It will be appreciated that since data acquisition, image analysis, and position analysis are constantly occurring during the length of any given imaging session that the tactile feedback provided in response to probe positioning may also be provided relatively continuously. For example, the probe may constantly be emitting feedback, and the type emitted may change as the user approaches and arrives at the desired position. This may present as a feedback parameter (intensity of vibration, for example), that gradually changes as the position becomes closer to the desired position.

Returning to 206, if the probe is in the correct position, then the method continues to 214. At 214, a first tactile feedback is provided through the probe. As introduced above, and described in greater detail below, different types of tactile feedback are meant to impart different messages to the user. “First tactile feedback” means only that a first correct signal has been output to the user that indicates to the user that the probe is in the desired position and does not need to be moved further before acquiring an image. A protocol for a single ultrasound procedure may contain a number of scan planes, in which case the user will receive a number of “first tactile feedback” signals, one with each new position the probe goes to. The type of tactile feedback indicating correct positioning of the probe may present in a mode different to that of incorrect feedback. For example, correct placement of the probe may present as force feedback around the entirety of the probe circumference in the hand held portion of the probe, while incorrect placement is indicated by vibration. Because the tactile feedback signal does not need to impart a direction to move the probe, the correct signal need not necessarily be localized to one portion of the probe. In an alternative embodiment, the first tactile feedback may present as one uniform strong vibration. In yet another embodiment, correct positioning may present as the absence of tactile feedback output. That is, the probe may constantly be emitting tactile feedback as the user moves the probe toward the desired position, but stop emitting feedback once the probe is in the correct position. In this way, correct positioning is imparted to the user by the absence of a tactile feedback signal. There may be many possible combinations of feedback mode and durations/patterns/strengths of that output mode. However, regardless of the specific combination of feedback mode and tactile feedback type, the first tactile feedback provided through the probe is unique to the other types of emitted tactile feedback described herein with reference to FIGS. 2 and 3 (e.g., second, third, etc.), so that the user may infer they are in the correct position to continue with ultrasound procedure.

In one embodiment, the user is only attempting to capture the image and/or video of one ROI from one scan plane, and having been provided the first tactile feedback signal at 214, the user may now capture the image and/or video of the desired region of interest (i.e., anatomical feature to be imaged), and move on to 226. At 226, the final image and/or video captured is stored. The image and/or video may be stored in the system memory, or a remote server, to be viewed at a later time. Having stored said image and/or video, the method comes to an end. In an alternative embodiment, the type of ultrasound modality used (e.g., Doppler) may determine if the user pursues some or all of the optional methodology depicted in 216-224. For example, regardless of whether the user is capturing one or more scan planes, the user may follow the methods at 216-218 because the user is capturing a video with a lengthy acquisition time in Doppler mode. The user may follow the steps at 216-218 (described in greater detail below) to acquire the desired video, and then, based on whether the user is following a protocol scanning multiple planes or not, the user may or may not perform the methods at 220-224. In an alternative embodiment, the user must image more than one ROI, more than one scan plane, or both (necessitating capture of more than one image and/or video), and so optional methodology is provided and explained below with reference to the methods at 216-224.

At 216, the methodology includes determining via the controller whether acquisition time is complete. For example, if the user was seeking to measure a kidney, a screen shot may suffice, and so acquisition time may be short. Comparatively, if a Doppler sample volume is being measured, the user may need to record a video, in which case acquisition time may be longer. The user may have previously signaled to the system controller at 216 that acquisition had begun. Depending on system operating conditions, while the acquisition is occurring, the system then determines if acquisition time is complete (i.e., determines if enough data has been recorded for the test being performed). If acquisition time is not complete the methodology moves to 218. At 218 the system continues acquiring data (i.e., storing an image and/or video) before returning to 216, and repeating the acquisition assessment. If acquisition time is still incomplete then the methodology will continue cycling between 216 and 218, until acquisition time is complete, and the methodology proceeds to 220.

At 220, the method includes providing a second tactile feedback indicating the scan is complete. Once the system has determined that enough data has been acquired for the protocol being performed, the controller then sends one or more signals to the actuators of the probe to output a unique tactile feedback signal, herein referred to as a “second tactile feedback” signal, to impart to the user that acquisition is complete and it is now okay to move the probe. In an alternative embodiment, the tactile feedback that may be emitted by the system is limited only to position feedback (i.e., signals strictly limited to correct or incorrect positioning), and is therefore incapable of signaling that a scan is complete. Alternative methods for signaling to the user that the required image and/or video has been stored and it is now safe to move the probe may include audio or visual feedback on the probe and/or display device.

At 222, the method includes determining whether the protocol is complete. The user, the system controller, or both (depending on system software, system operating conditions, user preference etc.) may determine the protocol is complete. Returning to the previous example in 216, a kidney may be imaged for measurement purposes. While the acquisition time may be short because only a screen shot is desired, the measurement protocol may require that the kidney be imaged from multiple planes of view (i.e., multiple scan planes). Should more imaging have to be done, the method continues to 224 where the system controller, user, or both, determines the next desired scan plane in the protocol. This may require the user to select a scan plane from a drop down menu on the display in order to covey to the system which scan plane is being pursued next. In another embodiment, the controller may have received a protocol from the user including a series of scan planes within the protocol before starting the protocol. In an alternative embodiment, the imaging system may already know which scan plane is being pursued next because the protocol is within the software of the program and scan planes are performed in a specific order. Once the system controller has determined the next desired scan plane in the protocol the methodology returns to 204, and the process begins again to accurately position the probe to acquire the newly determined scan plane. At 222, if it is determined that the protocol is complete, then the method continues to 226. At 226, the system stores final image(s) and/or video(s). Storing the image data may take place in a memory component of the system (such as the memory 120 in FIG. 1), so that it may be viewed for analysis at a later time. Analysis may take place immediately after storing the image, or pulled up a later date to compare with past/future images for temporal analysis (e.g., pregnancy growth, recession and remission of tumor, etc.).

In this way, the method 200 allows the system to impart information about correct or incorrect placement of a probe to the user via tactile feedback, in order to increase the accuracy and ease of positioning the probe. As a result, the acquired images may increase a medical professional's ability to analyze the images and make a diagnosis based on the acquired images.

FIG. 3 is a flow chart of a method 300 for a user utilizing a tactile feedback emitting probe of an imaging system (such as the imaging system 100 and probe 106 shown in FIG. 1) in order to increase probe placement accuracy during an imaging protocol. Method 300 is from the point of view of the user, who is only responding to probe tactile feedback, the patient, and the image on the display screen. Thus, method 300 may be an example of how the user interacts with an imaging system capable of providing tactile feedback via a probe, as described above with respect to FIG. 2 which illustrates a method carried out by the imaging system controller. As an example, method 300 may be a set of user instructions stored in a memory of the system controller and presented to the user during system operation via a user interface/display screen. It will be appreciated that prior to beginning method 300, the user will have already communicated to the system which protocol is being implemented (for example, a 3 vessel or PAV view of the heart, in B-mode).

Method 300 begins at 304, where the user moves the probe on the patient. For example, the user interface of the imaging system may prompt the user to position the probe on the patient or tissue to be scanned. Specifically, the user may place the probe on the patient in an area corresponding to a desired ROI to be scanned. The correct area for any given protocol and/or ROI will be familiar to those skilled in the art. The user may place the probe in an initial ballpark position, and begin moving toward the desired final position. During this period the imaging system is constantly acquiring ultrasound data, analyzing the incoming image data (and in one embodiment, data from internal motion sensors) to determine if the probe is in the desired position or not. It will be appreciated that in one embodiment, where the optional motion sensor data is not analyzed at the system controller level, the system may still be able to determine if the probe is in the desired position from image-analysis alone via image comparison with stored images, or independent image analysis for anatomical landmarks, yielding a binary result of “match” or “no match”. Once the user has begun moving the probe on the patient, the method continues to 306.

At 306, the user receives tactile feedback through the probe. The type of tactile feedback will impart to the user if the probe is in the correct position or not. Presumably, the first position the user places the probe in is not the desired final position, owing to the slight variances of each patient's anatomy, as well as skill level of the user. Therefore, it is likely that the user, upon moving the probe, will not receive the first tactile feedback signal (indicating correct positioning), but instead receive a third tactile feedback signal (indicating which direction to move the probe to approach the desired position). Note that “third” tactile feedback is only meant to impart that this is a unique signal that occurs when the probe is in an incorrect location, it is not meant to imply a numerical order of signal outputs. Furthermore, the “third” tactile feedback may be emitted multiple times, appearing to the user as an on-going signal, or as intermittent signals. The type of tactile feedback may present in the same or different mode as that of the correct tactile feedback signal, such as thermal (heat, cooling), vibrational, force feedback, etc. If the same mode is presented, say vibrational, then the output pattern will be different from that of the correct tactile feedback signal (described as first tactile feedback at 214 in FIG. 2). For example, the user may have previously communicated to the system that they are attempting to image a fetal heart. The user may have the probe in the incorrect position (perhaps over the fetus head instead of chest) and in response the user receives a vibrational signal from the actuators of the probe (e.g., the third tactile feedback signal), which indicates to the user the direction the probe needs to move to acquire the desired image. For example, assigning arbitrary cardinal directions wherein the fetus head is north and feet south, the user may receive vibrational signals from the southern probe actuators, which imparts to the user that the probe needs to move south. The strength of the signal may further impart to the user how far off the probe is, whereby a strong vibrational output means the probe is several inches away from the correct location, and a weak vibrational signal means the probe is very close to the correct location. It will be appreciated that activating regions of actuators at a time is only one way of imparting correctional signals to the user. In alternative embodiments, different modes of outputs may confer which direction the user needs to move the probe. Furthermore, variations of a single mode (i.e. strength of signal, pattern, duration, etc.) may also communicate correctional information to the user, so that the probe may be moved in a direction nearer to that of the ROI. Once the third tactile feedback has been provided in the direction of correct location of an ROI, the user then moves the probe according to the signal received (i.e., specific region of actuators emit signal and user moves probe in direction of actuator signal, in one embodiment) and the method continues to 307.

At 307, the user moves (e.g., such as rotating, tilting or linear movement) the probe on the patient based on the received tactile feedback. At 307, the type of feedback the user will have received will be of the third variety (due to the improbability of correct probe placement straightaway), instructing the user which way to move the probe to reach the desired position for a given protocol. Third tactile feedback will prompt the user to make a translational or rotational movement to move the probe to the desired location. In some embodiments, the third tactile feedback will be region specific on the probe, wherein only a subset of the actuators emit tactile feedback, thereby directing the user to move the probe in a specific direction corresponding to the final desired location. For example, the user may have placed the probe an inch “north” of the desired location, in this instance the actuators on the “south” end of the probe would emit a tactile feedback signal, which the user would interpret as a signal to move the probe “south”. As previously mentioned the strength, intensity, duration, etc. of the tactile feedback may present differently depending on how far off the probe is from the final desired location, however these slight variations would still be interpreted as a third tactile feedback signal. It will be appreciated that while 304, 306 and 307 are depicted as separate components of a methodology, they may all be happening almost simultaneously, and furthermore, continuously, until the desired probe position is arrived at. In an exemplary embodiment the user is moving the probe, and receiving feedback in a continuous manner, which brings the methodology to 308.

At 308, the user will determine if they have received first tactile feedback indicating correct probe position. The user may be receiving tactile feedback continuously while attempting to place the probe in the desired position. The user will likely have been utilizing a combination of the physical third tactile feedback signal and visual ultrasound image to reach the desired position (i.e., location and orientation). In this way, the user must determine with each tactile feedback signal received if the feedback is of the third (instructive) or first (conformation of correct positioning) variety. If the probe is still not in the correct position then the method will continue to 310. At 310, the user continues to move the probe on/in the patient based on received tactile feedback. The method at 310 may follow the methods of 304-307. Having moved the probe and received tactile feedback as previously described, the method returns to 308, where the user again determines if the tactile feedback received indicates correct positioning. The methodology will cycle between 308 and 310, until the probe is in both the correct location and orientation for acquiring the desired ROI and scan plane. Having received the first tactile feedback indicating correct position (described in 214 of FIG. 2), the user then instructs the system to capture the necessary image(s) and/or video(s) at the given position. In an alternative embodiment, the system may automatically capture the necessary image(s) and/or video(s). The method may then either continue to 318 and end, or continue to optional methodologies presented in 312-316, before also continuing to 318 and ending in an identical or similar fashion.

At 312, the user holds the probe in position until receiving second tactile feedback via the probe. Prior to receiving the second tactile feedback signal the system will be acquiring ultrasound data that will be displayed as an image(s) and/or video(s). Once the image(s) and/or video(s) are acquired in the correct position successfully, the user will receive the second tactile feedback signal, indicating to the user that the necessary image(s) and/or video(s) have been captured and that it is now okay to move the probe. It will be appreciated that after the first tactile feedback is received through the probe, the system may automatically capture the image, or the user may manually initiate image capture, depending on system operating conditions, software, etc. The way that second tactile feedback may present is previously described at 222 in FIG. 2. To summarize, the mode may be the same or different from the first or third tactile feedback modes, and may present as a subset of or all of the actuators at once. The most important factor is that the second tactile feedback signal is distinguishable from the other tactile feedback signals, so that the message it means to impart is unmistakable to the user (i.e., image has been captured from correct position and it is now okay to move the probe). Once the user receives this second tactile feedback signal the method continues to 314.

At 314, the user, or system controller, or both determines if the desired imaging protocol is complete. The protocol may require multiple images and/or videos in multiple positions, or multiple images and/or videos from the same position at multiple scan planes, or a combination of both. If the protocol is incomplete the method continues to 316. At 316, the user moves the probe to the next scan plane in the protocol and positions the probe on the patient accordingly. This may involve moving the probe to an entirely new location on the patient, or merely adjusting the angle of the probe to alter orientation. As one example, the method at 316 may include prompting the user (e.g., via a user interface of the system) to move the probe to the next scan plane in the protocol. For example, the system may present the next desired scan plane to the user. After 316, the method returns to 304 to begin again, where it is followed as previously described until once again returning to 314. If the protocol is still incomplete the loop repeats, and this continues any number of times until all image(s) and/or video(s) for the specified protocol are complete, and the method proceeds to 318.

At 318, the user may save and/or analyze the displayed image(s) and/or video(s). This may involve one image if the protocol required only one scan plane, or many images at once or in succession if the protocol required multiple scan planes, or possibly video if the protocol required it. At this point in the methodology the user may decide to place image(s)/video(s) in a folder, annotate the image(s)/video(s), analyze the image(s)/video(s), or perform any number of actions related to the placement, marking, analysis, sharing, storing, etc. of the image(s)/video(s). Having done this, method 300 comes to an end.

In this way, tactile feedback is output to a user via a probe of an imaging system depending on the probe's position. The system controller may have a plurality of image-analysis modules capable of analyzing the incoming data acquired by the probe to identify anatomical structures and/or landmarks. The image-analysis modules may additionally allow the system controller to determine if the probe is in the correct/incorrect position. Furthermore, the probe may be outfitted with internal sensors that can communicate the probe's movement to the system controller, where it can be analyzed alongside the incoming ultrasound data to determine how much and what type of probe movement resulted in an image change. A tactile feedback signal may be output to the user corresponding with the probe's position (i.e., different tactile feedback signals depending on correct/incorrect position). The tactile feedback emitted to the user via actuators of the probe may be based on correct position of the probe, incorrect position of the probe (incorrect location, incorrect orientation, or both), or as indication of completed image/video capture. The correct or incorrect position may be based on a desired positioning of the probe to acquire an image of a desired region of interest and scan plane. The type of tactile feedback emitted via the probe may come from one or more sets of actuators, and may present as one or more modes (e.g., thermal, vibrational, force feedback, etc.). The tactile feedback emitted from the probe may also vary according to signal strength/duration/pattern/etc. As a result, the user may feel the tactile feedback and infer whether the probe is in the desired position.

A technical effect of outputting tactile feedback via a probe that corresponds to the probe's position is allowing a user, especially an inexperienced user, to more quickly and accurately position the probe for medical imaging. For example, a user may be sure that the probe is in the correct location, but unsure if it is being held at the correct orientation to acquire the desired scan plane. To circumvent this issue the user may have previously been forced to take multiple images from multiple orientations, call in another user for a second opinion, etc. By receiving tactile feedback specific to positioning of the probe, the user is not only able to acquire the desired medical image, but able to do so in a more timely manner. Furthermore, an embodiment where the tactile feedback emitted by the probe guides the user toward the desired final position, increases efficiency by eliminating time the user may have spent trying to find the desired probe position.

In one embodiment, a method of ultrasound imaging comprises acquiring ultrasound data with a probe, generating an image based on the ultrasound data, determining whether the probe is in a desired position to acquire a desired region of interest, and providing a first tactile feedback through the probe in response to a determination that the probe is in the desired position. The first tactile feedback may be one or more of a vibration, force feedback, and thermal output that indicates the probe in is the correct position. Determining whether the probe is in the desired position to acquire the desired region of interest may include performing image analysis of the generated image to determine whether the generated image substantially matches an expected image for the desired region of interest. In one embodiment, performing the image analysis may include one or more of comparing the generated image to a stored image of the desired region of interest stored in a memory of an ultrasound imaging system and/or detecting anatomical landmarks in the generated image and matching the detected anatomical landmarks to a geometrical 3D model of the region of interest stored in the memory. In another embodiment, determining whether the probe is in the desired position to acquire the desired region of interest may be further based on outputs of one or more sensors disposed within the probe.

The methodology may include, in response to a determination that the probe is not in the desired position, providing a third tactile feedback through the probe, different than the first tactile feedback. Furthermore, the third tactile feedback may indicate a direction of a desired position of the desired region of interest and a desired scan plane. In one embodiment, the methodology may include, determining the third tactile feedback based on a desired direction of movement of the probe, where determining the desired direction of movement of the probe includes performing image analysis of the generated image. For example, providing the third tactile feedback through the probe may include actuating a subset of actuators within the probe to output tactile feedback, where the subset of actuators are positioned within a region of the probe proximate to the desired direction of movement of the probe.

The methodology may also include, after providing the first tactile feedback, continuing to acquire ultrasound data and, after a duration, providing a second tactile feedback indicating a scan of the desired region of interest in a first scan plane is complete and storing the acquired images in a memory of the ultrasound system. The methodology may further include increasing or decreasing the intensity of the third tactile feedback as the probe moves closer to the desired position.

In one embodiment, in response to an ultrasound protocol being complete, after providing the second tactile feedback, the methodology may include generating an updated image based on the acquired ultrasound data and displaying the generated updated image on a display. In another embodiment, a method comprises, in response to an ultrasound protocol not being complete, after providing the second tactile feedback, determining a second scan plane in the ultrasound protocol, acquiring ultrasound data and generating a second image, and providing tactile feedback through the probe to indicate a desired positioning of the probe for acquiring the second scan plane of the desired region of interest.

As another embodiment, a method of ultrasound imaging may comprise determining a change in position of an ultrasound probe and a resulting change in an image generated based on data acquired with the probe, and providing a first tactile feedback though the ultrasound probe to indicate a direction of a desired region of interest based on the determined change in position of the ultrasound probe and the resulting change in the image. The first tactile feedback provided through the ultrasound probe may include outputting one or more of a first vibration, force feedback, or thermal output via the ultrasound probe to indicate a desired direction of one or more of translational, pivotal, and rotational movement to move the probe toward the desired region of interest.

The method may further include, providing one or more of a visual feedback via a display and an audio feedback, in addition to providing the first tactile feedback to indicate the direction of the desired region of interest. In one embodiment, the method further comprises determining a desired direction of movement of the probe based on the determined change in position of the ultrasound probe and the resulting change in the image, as well as a comparing of the image to data stored within a memory of an ultrasound system, where the stored data includes one or more of stored image data corresponding to the desired region of interest and a computerized anatomical model of the desired region of interest.

In one embodiment, determining the desired direction of movement of the probe is further based on outputs from one or more motion sensors of the ultrasound probe indicating an amount of movement in the ultrasound probe that resulted in the change in the image. In another embodiment, providing the first tactile feedback through the probe may include actuating a subset of actuators to provide the first tactile feedback through the probe, where the subset of actuators are positioned at a region of the probe proximate to the determined desired direction of movement of the probe.

In yet another embodiment, an ultrasound imaging system comprises a probe, a user interface, and a processor in communication with the user interface and probe. The processor includes a non-transitory memory with instructions for receiving a desired scan plane and region of interest, acquiring first ultrasound data with the probe in a first position, generating a first image based on the first ultrasound data, automatically determining whether the probe is positioned in a correct position to acquire the desired scan plane and region of interest based on the generated first image and image data stored in the memory, and in response to the probe not being in the correct position, providing a first tactile feedback through the probe.

In one example, the system's instructions may further include instructions for acquiring second ultrasound data with the probe in a second position, different than the first, generating a second image based on the second ultrasound data, and in response to the probe being in the correct position to acquire the desired scan plane and region of interest, providing a second tactile feedback through the probe, different than the first. Furthermore, the instructions may include instructions for displaying the generated second image on a display of the ultrasound imaging system. Methodology of the system described may include, providing the first tactile feedback through the probe including outputting a signal via one or more probe actuators indicating a direction of the correct position via the probe, wherein the direction of the correct position is determined based on the generated first image, a third image generated prior to the first image, and a motion of the probe between acquisition of the third and first images.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method of ultrasound imaging, comprising:
acquiring ultrasound data with a probe;
generating an image based on the ultrasound data;
determining whether the probe is in a desired position to acquire a desired region of interest; and
providing a first tactile feedback through the probe in response to a determination that the probe is in the desired position.

2. The method of claim 1, wherein the first tactile feedback is one or more of a vibration, force feedback, and thermal output that indicates the probe is in the correct position.

3. The method of claim 1, wherein determining whether the probe is in the desired position to acquire the desired region of interest includes performing image analysis of the generated image to determine whether the generated image substantially matches an expected image for the desired region of interest.

4. The method of claim 3, wherein performing the image analysis includes one or more of comparing the generated image to a stored image of the desired region of interest stored in a memory of an ultrasound imaging system and detecting anatomical landmarks in the generated image and matching the detected anatomical landmarks to a geometrical model of the region of interest stored in the memory.

5. The method of claim 3, wherein determining whether the probe is in the desired position to acquire the desired region of interest is further based on outputs of one or more motion sensors disposed within the probe.

6. The method of claim 1, furthering comprising, in response to a determination that the probe is not in the desired position, providing a third tactile feedback through the probe, different than the first tactile feedback, wherein the third tactile feedback indicates a direction of a desired position for the desired region of interest and a desired scan plane.

7. The method of claim 6, further comprising determining the third tactile feedback based on a desired direction of movement of the probe, where determining the desired direction of movement of the probe includes performing image analysis of the generated image.

8. The method of claim 7, wherein providing the third tactile feedback through the probe includes actuating a subset of actuators within the probe to output tactile feedback, where the subset of actuators are positioned within a region of the probe proximate to the desired direction of movement of the probe.

9. The method of claim 6, further comprising, after providing the first tactile feedback, continuing to acquire ultrasound data and, after a duration, providing a second tactile feedback indicating a scan of the desired region of interest in a first scan plane is complete and storing the acquired images or videos in a memory of the ultrasound imaging system.

10. The method of claim 6, wherein an intensity of the third tactile feedback increases or decreases as the probe moves closer to the desired position.

11. A method of ultrasound imaging, comprising:
determining a change in position of an ultrasound probe and a resulting change in an image generated based on data acquired with the probe; and
providing a first tactile feedback through the ultrasound probe to indicate a direction of a desired region of interest based on the determined change in position of the ultrasound probe and the resulting change in the image.

12. The method of claim 11, wherein providing the first tactile feedback through the ultrasound probe includes outputting one or more of a first vibration, force feedback, or thermal output via the ultrasound probe to indicate a desired direction of one or more of translational, pivotal, and rotational movement to move the probe toward the desired region of interest.

13. The method of claim 11, further comprising providing one or more of a visual feedback via a display and an audio feedback in addition to providing the first tactile feedback to indicate the direction of the desired region of interest.

14. The method of claim 11, further comprising determining a desired direction of movement of the probe based on the determined change in position of the ultrasound probe and the resulting change in the image and a comparing the image to data stored within a memory of an ultrasound system, where the stored data includes one or more of stored image data corresponding to the desired region of interest and a computerized anatomical model of the desired region of interest.

15. The method of claim 14, wherein the determining the desired direction of movement of the probe is further based on outputs from one or more motion sensors of the ultrasound probe indicating an amount of movement in the ultrasound probe that resulted in the change in the image.

16. The method of claim 14, wherein providing the first tactile feedback through the probe includes actuating a subset of actuators to provide the first tactile feedback through the probe, where the subset of actuators are positioned at a region of the probe proximate to the determined desired direction of movement of the probe.

17. An ultrasound imaging system, comprising:
a probe;
a user interface;
a processor in communication with the user interface and probe and including a non-transitory memory with instructions for:
receiving a desired scan plane and region of interest;
acquiring first ultrasound data with the probe in a first position;
generating a first image based on the first ultrasound data;
automatically determining whether the probe is positioned in a correct position to acquire the desired scan plane and region of interest based on the generated first image and image data stored in the memory; and
in response to the probe not being in the correct position, providing a first tactile feedback through the probe.

18. The system of claim 17, wherein the instructions further include instructions for acquiring second ultrasound data with the probe in a second position, different than the first, generating a second image based on the second ultrasound data, and in response to the probe being in the correct position to acquire the desired scan plane and region of interest, providing a second tactile feedback through the probe, different than the first, and wherein the instructions further include instructions for displaying the generated second image on a display of the ultrasound imaging system.

19. The system of claim 17, wherein providing the first tactile feedback through the probe includes outputting a signal via one or more probe actuators indicating a direction of the correct position via the probe.

20. The system of claim 19, wherein the direction of the correct position is determined based on the generated first image, a third image generated prior to the first image, and a motion of the probe between acquisition of the third and first images.
